(19) United States
(12) Patent Application Publication
(10) Pub. No.: US 2020/0283381 A1
(43) Pub. Date: Sep. 10, 2020

(54) SOLID STATE FORMS OF ELAFIBRANOR

(71) Applicant: TEVA PHARMACEUTICALS INTERNATIONAL GMBH, Jona (CH)

(72) Inventors: Ivana LANDEKA, Zagreb (HR); Dijana Skalec SAMEC, Jastrebarsko (HR)

(21) Appl. No.: 16/651,105

(22) PCT Filed: Nov. 16, 2018
(86) PCT No.: PCT/US2018/061417

§ 371 Date: Mar. 26, 2020

Related U.S. Application Data
(60) Provisional application No. 62/587,094, filed on Nov. 16, 2017.
(60) Provisional application No. 62/610,583, filed on Dec. 27, 2017.
(60) Provisional application No. 62/624,938, filed on Feb. 1, 2018.

(51) Int. Cl. C07C 323/62; A61K 9/00

(52) U.S. Cl. CPC C07C 323/62; C07B 2200/13; A61K 9/0053

(57) ABSTRACT

Disclosed are solid state forms of Elafibranor and salts thereof, processes for preparation thereof and pharmaceutical compositions thereof.

### FIELD OF THE INVENTION

The present disclosure relates to solid state forms of Elafibranor and salts thereof, processes for preparation thereof and pharmaceutical compositions thereof.

### BACKGROUND OF THE INVENTION

Elafibranor has the chemical name 2-(2,6-dimethyl-4-{3-[4-(methylsulfanyl)phenyl]-3-oxoprop-1-en-1-yl}phenoxy)-2-methylpropanoic acid. Elafibranor has the following chemical structure:

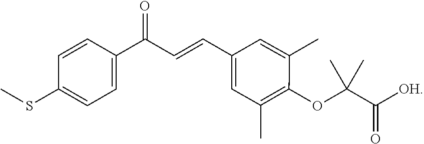 [SMILES: CSc1ccc(C(=O)/C=C/c2cc(C)c(OC(C)(C)C(=O)O)c(C)c2)cc1]

Elafibranor is an oral once-daily administered molecule, acting via dual peroxisome proliferator-activated (PPAR) alpha/delta pathways developed by Genfit to treat, in particular, nonalcoholic steatohepatitis (NASH). Elafibranor is believed to address multiple facets of NASH, including inflammation, insulin sensitivity, lipid/metabolic profile, and liver markers.

Elafibranor and process for preparation thereof are known from WO2004/05233 and WO2005/005369. Crystalline forms of Elafibranor are known from WO2018133705 and IPCOM00025280.

Polymorphism, the occurrence of different crystal forms, is a property of some molecules and molecular complexes. A single compound, like Elafibranor, may give rise to a variety of polymorphs having distinct crystal structures and physical properties like melting point, thermal behaviors (e.g. measured by thermogravimetric analysis—“TGA”, or differential scanning calorimetry—“DSC”), powder X-ray diffraction (PXRD) pattern, infrared absorption fingerprint,

Raman absorption fingerprint, and solid state (¹³C-) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms and solvates of a pharmaceutical product can provide materials having desirable processing properties, such as ease of handling, ease of processing, storage stability, and ease of purification or as desirable intermediate crystal forms that facilitate conversion to other salts or polymorphic forms. New salts, polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for solid state forms (including solvated forms) of Elafibranor and salts thereof, in particular as polymorphically pure material.

### SUMMARY OF THE INVENTION

The present disclosure relates to solid state forms of Elafibranor and salts thereof, to processes for preparation thereof, and to pharmaceutical compositions comprising these solid state forms.

The present disclosure also provides uses of the solid state forms of Elafibranor and salts thereof for preparing other solid state forms of Elafibranor, Elafibranor salts and solid state forms thereof.

In another embodiment, the present disclosure encompasses the above described solid state forms of Elafibranor and salts thereof for use in the preparation of pharmaceutical compositions and/or formulations, preferably for the treatment of nonalcoholic steatohepatitis (NASH).

In another embodiment the present disclosure encompasses the use of the above described solid state form of Elafibranor and salts thereof for the preparation of pharmaceutical compositions and/or formulations.

The present disclosure further provides pharmaceutical compositions comprising the solid state forms of Elafibranor and salts thereof according to the present disclosure.

In yet another embodiment, the present disclosure encompasses pharmaceutical formulations comprising the above described solid state forms of Elafibranor and salts thereof and at least one pharmaceutically acceptable excipient, preferably for oral administration in a dosage forms such as tablets, capsules etc.

The present disclosure encompasses processes to prepare said pharmaceutical formulations of Elafibranor comprising combining the above solid state forms and at least one pharmaceutically acceptable excipient.

The solid state forms as defined herein, as well as the pharmaceutical compositions or formulations of the solid state form of Elafibranor and salts thereof, can be used as medicaments, particularly for the treatment of nonalcoholic steatohepatitis (NASH).

The present disclosure also provides methods of treating nonalcoholic steatohepatitis (NASH), comprising administering a therapeutically effective amount of the solid state form of Elafibranor and salts thereof of the present disclosure, or at least one of the above pharmaceutical compositions or formulations, to a subject suffering from nonalcoholic steatohepatitis, or otherwise in need of the treatment.

The present disclosure also provides uses of the solid state forms of Elafibranor and salts thereof of the present disclosure, or at least one of the above pharmaceutical compositions or formulations for the manufacture of a medicament for treating nonalcoholic steatohepatitis.

### BRIEF DESCRIPTION OF THE FIGURES

FIG. 1 shows a powder X-ray diffraction pattern (“powder XRD” or “PXRD”) of Elafibranor form I obtained in Example 1.

FIG. 2 shows a powder X-ray diffraction pattern (“powder XRD” or “PXRD”) of Elafibranor form II obtained in Example 2.

FIG. 3 shows a powder X-ray diffraction pattern (“powder XRD” or “PXRD”) of Elafibranor form III obtained in Example 3.

FIG. 4 shows a powder X-ray diffraction pattern (“powder XRD” or “PXRD”) of Elafibranor form IV.

FIG. 5 shows a powder X-ray diffraction pattern (“powder XRD” or “PXRD”) of Elafibranor form V.

FIG. 6 shows a powder X-ray diffraction pattern (“powder XRD” or “PXRD”) of Elafibranor form VI.

FIG. 7 shows a powder X-ray diffraction pattern (“powder XRD” or “PXRD”) of Elafibranor form VII.

FIG. 8 shows a DSC thermogram of Elafibranor form I.

FIG. 9 shows a DSC thermogram of Elafibranor form II.

### DETAILED DESCRIPTION OF THE INVENTION

The present disclosure relates to solid state forms of Elafibranor (crystalline and amorphous forms) and salts thereof, processes for preparation thereof and pharmaceutical compositions comprising said solid state forms.

The solid state forms of Elafibranor according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Elafibranor and salts thereof referred to herein as being characterized by graphical data “as depicted in” a Figure will thus be understood to include any crystal forms of the Elafibranor and salts thereof, characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or about 0% of any other forms of the subject compound as measured, for example, by PXRD. Thus, solid state of Elafibranor and Elafibranor salts, described herein as substantially free of any other solid state forms would be understood to contain greater than about 80% (w/w), greater than about 90% (w/w), greater than about 95% (w/w), greater than about 98% (w/w), greater than about 99% (w/w), or about 100% (w/w) of the subject solid state form of Elafibranor and Elafibranor salts. Accordingly, in some embodiments of the disclosure, the described solid state forms of Elafibranor and Elafibranor salts may contain from about 1% to about 20% (w/w), from about 5% to about 20% (w/w), or from about 5% to about 10% (w/w) of one or more other solid state forms of the same Elafibranor and Elafibranor salts.

As used herein, unless stated otherwise, PXRD peaks reported herein are preferably measured using CuK_{α} radiation, λ=1.5418 Å.

As used herein, unless stated otherwise, DSC data is obtained at a heating rate of 10° C./min.

As used herein, the term “isolated” in reference to solid state forms of Elafibranor and Elafibranor salts, of the present disclosure corresponds to solid state forms of Elafibranor and Elafibranor salts that are physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The crystal hydrate indicated by water analysis by Karl Fischer (KF) titration or by TGA analysis of this product is believed to have been produced as a result of water introduced from the atmosphere in which this material was processed, or by traces of water present in the solvents that were in contact with the material, or a combination of these factors.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

As used herein, and unless stated otherwise, the term “anhydrous” in relation to crystalline Elafibranor relates to a crystalline Elafibranor which does not include any crystalline water (or other solvents) in a defined, stoichiometric amount within the crystal. Moreover, an “anhydrous” form does not contain more than 1% (w/w) of either water or organic solvents as measured for example by TGA.

As used herein the term non-hygroscopic in relation to crystalline Elafibranor refers to less than 0.1% (w/w) of water absorption after 24 h exposure to 25° C./80% RH, determined according to European Pharmacopoeia 7.0, chapter 01/2008:51100. Water can be for example atmospheric water.

The present disclosure comprises crystalline Elafibranor:

(a) having a PXRD pattern with peaks at 7.8, 12.3, 13.5, 15.9 and 22.7 degrees 2-theta±0.2 degrees 2-theta;

(b) having a PXRD pattern with peaks at 7.8, 12.3, 13.5, 15.9 and 22.7 degrees 2-theta±0.2 degrees 2-theta and also having one, two, three or four additional peaks at 15.7, 16.3, 17.1, and 23.4 degrees 2-theta±0.2 degrees 2-theta;

(c) having a PXRD pattern as described in (a) or (b), and further characterized by one, two, three or four additional peaks at: 15.3, 21.4, 27.8 and 28.3 degrees 2-theta±0.1 degrees 2-theta;

(d) having a PXRD pattern as described in (a) or (b), and further characterized by one, two, three or four additional peaks at 21.2, 25.0, 27.6 and 28.1 degrees 2-theta±0.1 degrees 2-theta;

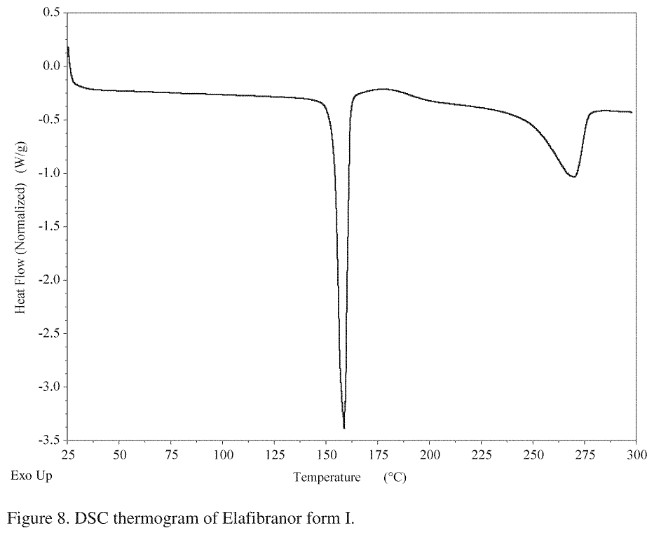

(e) having a PXRD pattern as described in (a), (b) or (c), and a melting point peak at about 154° C. to about 164° C., preferably at about 158° C. to about 160° C., and most preferably at about 159° C., as measured by DSC and preferably a DSC thermogram exhibiting an endothermic peak with onset in the range of about 140° C. to about 160° C., more preferably in the range of about 150° C. to about 158° C., in particular at about 154° C.; and particularly has a DSC thermogram substantially as depicted in FIG. 8;

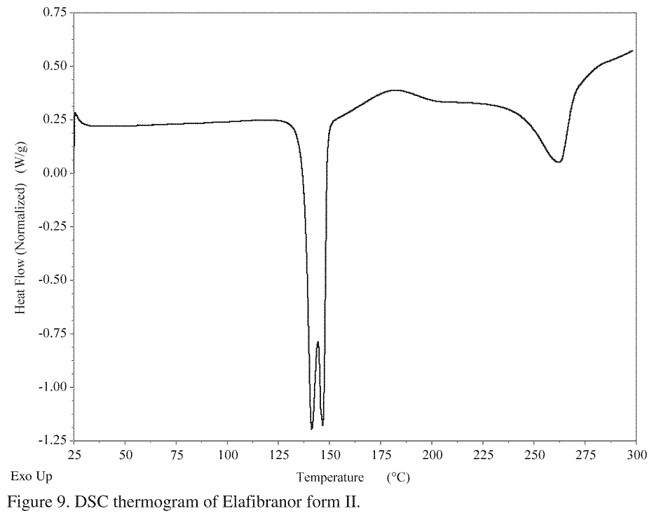

(f) having a PXRD pattern as described in (a), (b) or (d), and a DSC thermogram which exhibits an endothermic event with onset in the range of about 127° C. to about 155° C., preferably in the range of about 130° C. to about 150° C., and more preferably exhibits two consecutive endothermic events with onsets in the range of about 137° C. and about 145° C. as measured by DSC with heating rate of 10° C., and preferably a DSC thermogram substantially as depicted in FIG. 9

The crystalline Elafibranor as described above in (a)-(f) may be anhydrous. The crystalline Elafibranor may be substantially free of other solid state forms of Elafibranor. The crystalline Elafibranor is non-hygroscopic and is thermodynamically stable. Particularly, the crystalline Elafibranor shows less than 20%, less than 10%, less than 5%, less than 2%, less than 1%, or less than 0.5% conversion to any other solid state form of Elafibranor after exposure to conditions of up to 25° C./100% RH for at least 1 month, as measured by XRPD. In some embodiments, the conversion is 1%-20%, 1%-10% or 1%-5%.

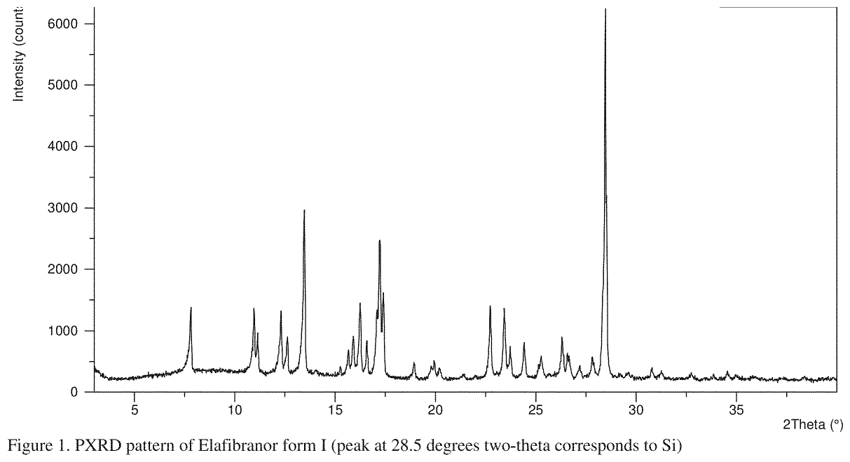

The present disclosure further comprises a crystalline form of Elafibranor designated as form I. The crystalline form I of Elafibranor can be characterized by data selected from one or more of the following: a PXRD pattern having peaks at 7.8, 16.3, 17.2, 24.4 and 28.3 degrees 2-theta±0.1 degrees 2-theta; a PXRD pattern as depicted in FIG. 1 and combinations of these data.

Crystalline form I of Elafibranor may be further characterized by the PXRD pattern having peaks at 7.8, 16.3, 17.2, 24.4 and 28.3 degrees 2-theta±0.1 degrees 2-theta, and also having one, two, three, four or five additional peaks at 11.2, 15.3, 21.4, 25.3 and 27.8 degrees 2-theta±0.1 degrees 2-theta; a DSC thermogram as depicted in FIG. 8 and combinations of these data.

Crystalline form I of Elafibranor may be characterized by each of the above characteristics alone/or by all possible combinations, e.g., by PXRD pattern having peaks at 7.8, 16.3, 17.2, 24.4 and 28.3 degrees 2-theta±0.1 degrees 2-theta and a PXRD pattern as depicted in FIG. 1.

Crystalline form I of Elafibranor may be polymorphically pure.

Crystalline form I of Elafibranor according to any of the above embodiments may be an anhydrous form.

Crystalline form I of Elafibranor according to any of the above embodiments is non-hygroscopic.

Crystalline form I of Elafibranor according to any of the above embodiments is thermodynamically stable. Melting peak of crystalline form I of Elafibranor is in the range of about 154° C. and about 164° C., for example about 158° C. and about 160° C., preferably at about 159° C. as measured by DSC. Crystalline form I of Elafibranor exhibits an endothermic peak with onset in the range of about 140° C. and about 160° C., for example in the range of about 150° C. and about 158° C., in particular at around 154° C. as measured by DSC with heating rate of 10° C. Thermodynamic stability in relation to crystalline Elafibranor form I refers to less than 20%, less than 10%, less than 5%, less than 2%, less than 1%, or less than 0.5% conversion of crystalline Elafibranor form I to any other solid state form of Elafibranor after exposure of form I to conditions of up to 25° C./100% RH for at least 1 month, as measured by XRPD. In some embodiments, the conversion is 1%-20%, 1%-10% or 1%-5%.

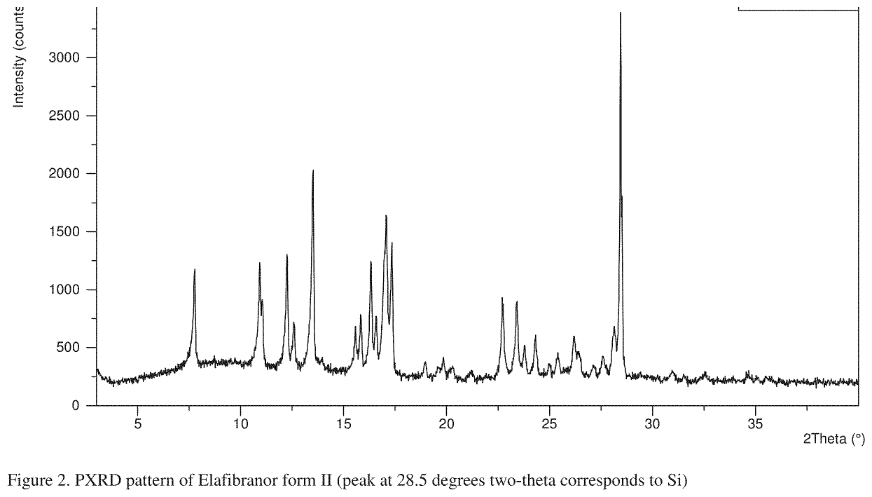

The present disclosure comprises a crystalline form of Elafibranor designated as form II. The crystalline form II of Elafibranor can be characterized by data selected from one or more of the following: a PXRD pattern having peaks at 10.9, 23.8, 24.3 and 28.1 degrees 2-theta±0.1 degrees 2-theta; a PXRD pattern as depicted in FIG. 2 and combinations of these data.

Crystalline form II of Elafibranor may be further characterized by the PXRD pattern having peaks at 10.9, 23.8, 24.3 and 28.1 degrees 2-theta±0.1 degrees 2-theta, and also having one, two, three or four additional peaks at 15.6, 21.2, 25.0 and 27.6 degrees 2-theta±0.1 degrees 2-theta; a DSC thermogram as depicted in FIG. 9 and combinations of these data.

Crystalline form II of Elafibranor may be characterized by each of the above characteristics alone/or by all possible combinations, e.g., by PXRD pattern having peaks at 10.9, 23.8, 24.3 and 28.1 degrees 2-theta±0.1 degrees 2-theta and a PXRD pattern as depicted in FIG. 2.

Crystalline form II of Elafibranor may be polymorphically pure.

Crystalline form II of Elafibranor according to any of the above embodiments may be an anhydrous form.

Crystalline form II of Elafibranor according to any of the above embodiments is non-hygroscopic.

Crystalline form II of Elafibranor exhibits an endothermic event with onset in the range of about 127° C. and about 155° C., for example in the range of about 130° C. to about 150° C., in particular crystalline form II of Elafibranor exhibits two consecutive endothermic events with onsets in the range of about 137° C. to about 145° C. as measured by DSC with heating rate of 10° C. Crystalline form II of Elafibranor according to any of the above embodiments is thermodynamically stable. Thermodynamic stability in relation to crystalline Elafibranor form II refers to less than 20%, 10%, 5%, 2%, 1%, or 0.5% conversion of crystalline Elafibranor form II to any other solid state form of Elafibranor after exposure of form II to conditions of up to 25° C./100% RH for at least 1 month, as measured by XRPD. In some embodiments, the conversion is 1%-20%, 1%-10% or 1%-5%.

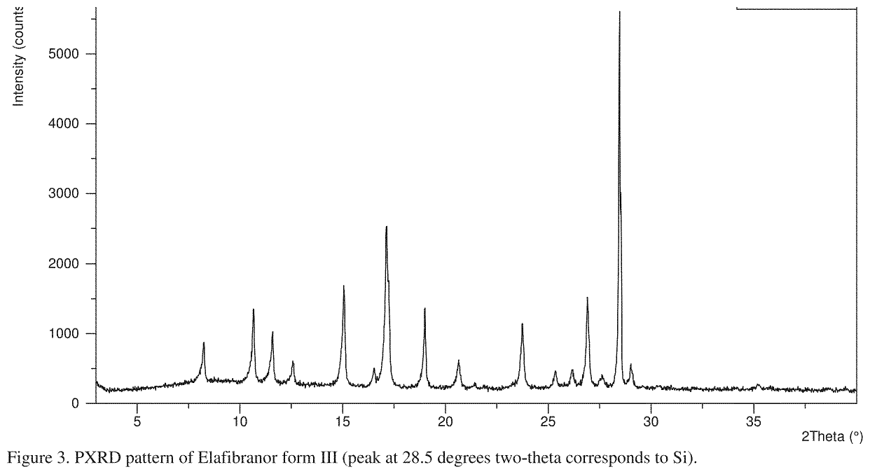

The present disclosure comprises a crystalline form of Elafibranor designated as form III. The crystalline form III of Elafibranor can be characterized by data selected from one or more of the following: a PXRD pattern having peaks at 8.3, 11.6, 19.0 20.7 and 23.7 degrees 2-theta±0.2 degrees 2-theta; a PXRD pattern as depicted in FIG. 3; or combinations of these data.

Crystalline form III of Elafibranor may be further characterized by the PXRD pattern having peaks at 8.3, 11.6, 19.0 20.7 and 23.7 degrees 2-theta±0.2 degrees 2-theta, and also having one, two, three, four or five additional peaks at 10.7, 15.1, 25.3, 26.9 and 29.0 degrees 2-theta±0.2 degrees 2-theta.

Crystalline form III of Elafibranor may be characterized by each of the above characteristics alone/or by all possible combinations, e.g., by PXRD pattern having peaks at 8.3, 11.6, 19.0 and 20.7 and 23.7 degrees 2-theta±0.2 degrees 2-theta and a PXRD pattern as depicted in FIG. 3.

Crystalline form III of Elafibranor may be polymorphically pure.

Crystalline form III of Elafibranor according to any of the above embodiments may be an anhydrous form.

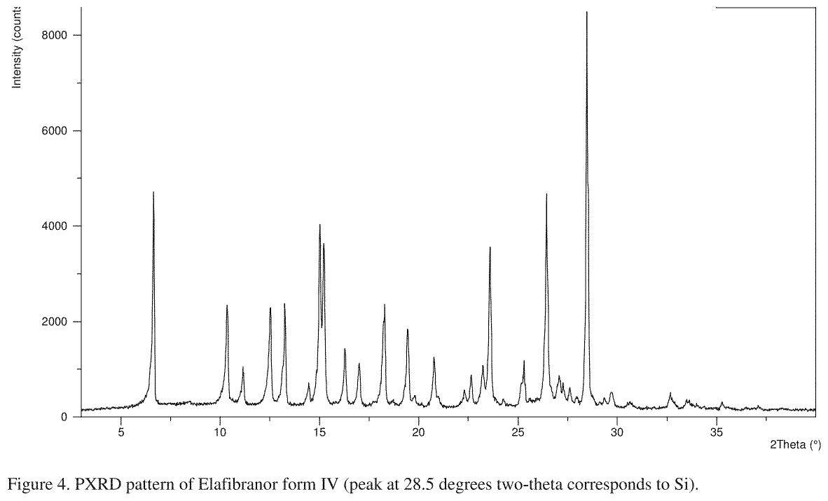

The present disclosure comprises a crystalline form of Elafibranor designated as form IV. The crystalline form IV of Elafibranor can be characterized by data selected from one or more of the following: a PXRD pattern having peaks at 6.6, 10.4, 18.3, 19.4 and 23.6 degrees 2-theta±0.2 degrees 2-theta; a PXRD pattern as depicted in FIG. 4; or combinations of these data.

Crystalline form IV of Elafibranor may be further characterized by the PXRD pattern having peaks at 6.6, 10.4, 18.3, 19.4 and 23.6 degrees 2-theta±0.2 degrees 2-theta, and also having one, two, three, four or five additional peaks at 12.5, 15.0, 15.2, 20.8 and 26.4 degrees 2-theta±0.2 degrees 2-theta.

Crystalline form IV of Elafibranor may be characterized by each of the above characteristics alone/or by all possible combinations, e.g., by PXRD pattern having peaks at 6.6, 10.4, 18.3, 19.4 and 23.6 degrees 2-theta±0.2 degrees 2-theta and a PXRD pattern as depicted in FIG. 4.

Crystalline form IV of Elafibranor may be polymorphically pure.

Crystalline form IV of Elafibranor according to any of the above embodiments may be acetate solvate.

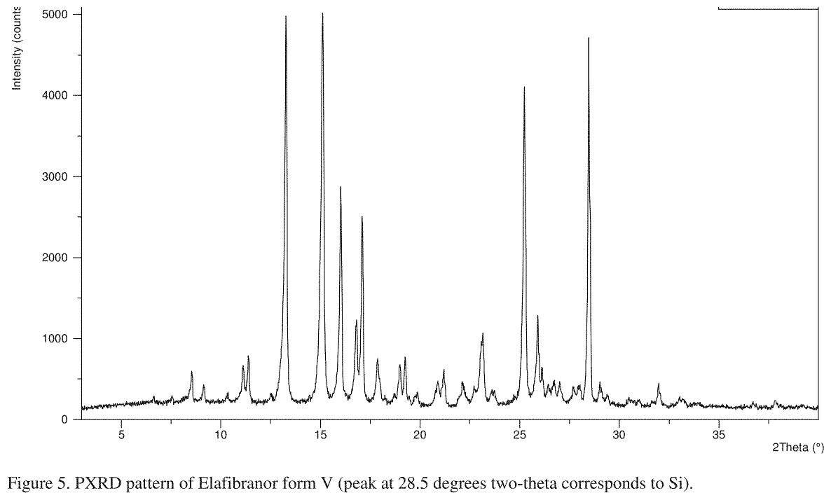

The present disclosure comprises a crystalline form of Elafibranor designated as form V. The crystalline form V of Elafibranor can be characterized by data selected from one or more of the following: a PXRD pattern having peaks at 13.3, 15.1, 16.0, 16.8 and 17.1 degrees 2-theta±0.2 degrees 2-theta; a PXRD pattern as depicted in FIG. 5; or combinations of these data.

Crystalline form V of Elafibranor may be further characterized by the PXRD pattern having peaks at 13.3, 15.1, 16.0, 16.8 and 17.1 degrees 2-theta±0.2 degrees 2-theta, and also having one, two, three, four or five additional peaks at 8.5, 11.4, 20.9, 21.2 and 25.9 degrees 2-theta±0.2 degrees 2-theta.

Crystalline form V of Elafibranor may be characterized by each of the above characteristics alone/or by all possible combinations, e.g., by PXRD pattern having peaks at 13.3, 15.1, 16.0, 16.8 and 17.1 degrees 2-theta±0.2 degrees 2-theta and a PXRD pattern as depicted in FIG. 5.

Crystalline form V of Elafibranor may be polymorphically pure.

Crystalline form V of Elafibranor according to any of the above embodiments may be dimethylacetamide solvate.

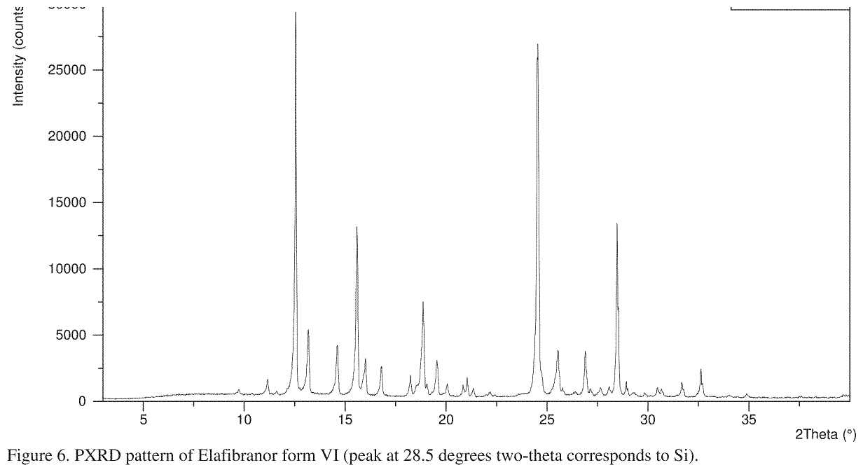

The present disclosure comprises a crystalline form of Elafibranor designated as form VI. The crystalline form VI of Elafibranor can be characterized by data selected from one or more of the following: a PXRD pattern having peaks at 12.6, 14.6, 15.6, 18.9 and 25.5 degrees 2-theta±0.2 degrees 2-theta; a PXRD pattern as depicted in FIG. 6; or combinations of these data.

Crystalline form VI of Elafibranor may be further characterized by the PXRD pattern having peaks at 12.6, 14.6, 15.6, 18.9 and 25.5 degrees 2-theta±0.2 degrees 2-theta, and also having one, two, three, four or five additional peaks at 19.5, 21.0, 24.5, 26.9 and 31.7 degrees 2-theta±0.2 degrees 2-theta.

Crystalline form VI of Elafibranor may be characterized by each of the above characteristics alone/or by all possible combinations, e.g., by PXRD pattern having peaks at 12.6, 14.6, 15.6, 18.9 and 25.5 degrees 2-theta±0.2 degrees 2-theta and a PXRD pattern as depicted in FIG. 6.

Crystalline form VI of Elafibranor may be polymorphically pure.

Crystalline form VI of Elafibranor may be N-methyl-2-pyrrolidone solvate.

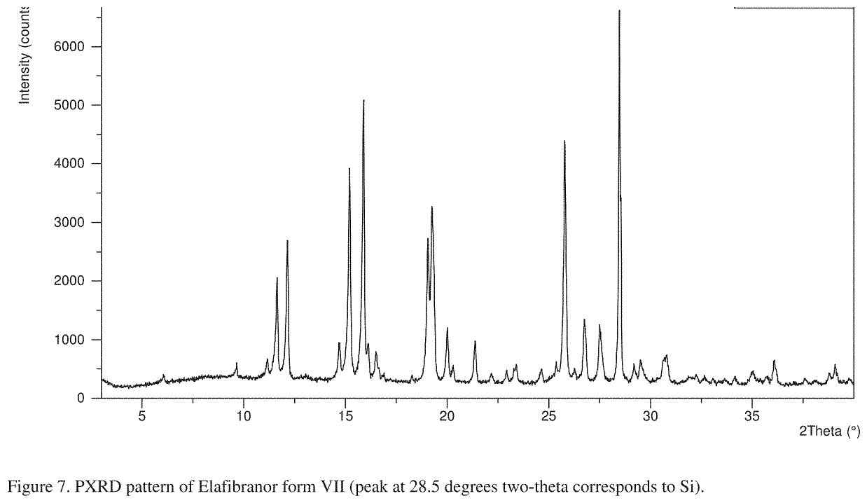

The present disclosure comprises a crystalline form of Elafibranor designated as form VII. The crystalline form VII of Elafibranor can be characterized by data selected from one or more of the following: a PXRD pattern having peaks at 6.1, 9.6, 12.1, 20.0 and 25.8 degrees 2-theta±0.2 degrees 2-theta; a PXRD pattern as depicted in FIG. 7; or combinations of these data.

Crystalline form VII of Elafibranor may be further characterized by the PXRD pattern having peaks at 6.1, 9.6, 12.1, 20.0 and 25.8 degrees 2-theta±0.2 degrees 2-theta, and also having one, two, three, four or five additional peaks at 14.7, 15.9, 19.1, 19.2 and 26.7 degrees 2-theta±0.2 degrees 2-theta.

Crystalline form VII of Elafibranor may be characterized by each of the above characteristics alone/or by all possible combinations, e.g., by PXRD pattern having peaks at 6.1, 9.6, 12.1, 20.0 and 25.8 degrees 2-theta±0.2 degrees 2-theta and a PXRD pattern as depicted in FIG. 7.

Crystalline form VII of Elafibranor may be polymorphically pure.

Crystalline form VII of Elafibranor may be a hydrate, in particular a monohydrate.

Crystalline form VII of Elafibranor according to any of the above embodiments is a hydrate having about 4% to about 5% (w/w) for example 4.5% (w/w) of water as measure by TGA.

The present disclosure also provides the use of the solid state forms of Elafibranor base and Elafibranor salts, for preparing other solid state forms of Elafibranor, Elafibranor salts and solid state forms thereof.

The present disclosure further encompasses processes for preparing Elafibranor salts or solid state forms thereof. The process comprises preparing the solid state form of the present disclosure, and converting it to other solid state form of Elafibranor. Alternatively, the process comprises preparing the solid state form of the present disclosure, and converting it to Elafibranor salt. The conversion can be done, for example, by a process comprising reacting the obtained Elafibranor with an appropriate base to obtain the corresponding base addition.

In another embodiment the present disclosure encompasses the above described solid state forms of Elafibranor and salts thereof, for use in the preparation of pharmaceutical compositions and/or formulations, preferably for the treatment of NASH.

In another embodiment the present disclosure encompasses the use of the above described solid state forms of Elafibranor and salts thereof, or combinations thereof, for the preparation of pharmaceutical compositions and/or formulations, preferably oral formulations, e.g. tablets or capsules.

The present disclosure further provides pharmaceutical compositions comprising the solid state forms of Elafibranor and salts thereof, or combinations thereof, according to the present disclosure.

In yet another embodiment, the present disclosure encompasses pharmaceutical formulations comprising at least one of the above described solid state forms of Elafibranor and salts thereof, and at least one pharmaceutically acceptable excipient.

The present disclosure encompasses a process to prepare said formulations of Elafibranor comprising combining at least one of the above solid state forms and at least one pharmaceutically acceptable excipient.

The solid state forms as defined herein, as well as the pharmaceutical compositions or formulations of Elafibranor can be used as medicaments, particularly for the treatment of NASH.

The present disclosure also provides a method of treating NASH, comprising administering a therapeutically effective amount of the solid state form of Elafibranor of the present disclosure, or at least one of the above pharmaceutical compositions or formulations, to a subject suffering from NASH or otherwise in need of the treatment.

The present disclosure also provides the use of the solid state forms of Elafibranor of the present disclosure, or at least one of the above pharmaceutical compositions or formulations for the manufacture of a medicament for treating NASH.

Having described the invention with reference to certain preferred embodiments, other embodiments will become apparent to one skilled in the art from consideration of the specification. The invention is further illustrated by reference to the following examples describing in detail the preparation of the composition and methods of use of the invention. It will be apparent to those skilled in the art that many modifications, both to materials and methods, may be practiced without departing from the scope of the invention.

### Analytical Methods

### Powder X-Ray Diffraction Pattern (“PXRD”) Method:

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Ã (Angstrom), X'Celerator (2.022° 20) detector. Scanning parameters: angle range: 3-40 deg., step size 0.0167, time per step 37 s, continuous scan.

The described peak positions were determined using silicon powder as an internal standard in an admixture with the sample measured. The position of the silicon (Si) peak was corrected to silicone theoretical peak: 28.45 degrees two theta, and the positions of the measured peaks were corrected respectively.

### Differential Scanning Calorimetry (DSC) Method:

DSC analysis was performed on TA Instruments Discovery DSC with heating rate of 10° C./min in the range of 25°−300° C. and purged with 50 ml/min of nitrogen. A hermetic aluminium, closed pan with hole was used, and the sample mass was about 2 mg.

### Thermogravimetric Analysis (TGA) Method:

Thermogravimetric analysis was carried out on TA Instruments Discovery TGA with heating rate of 10° C./min in the range of 25°−300° C. and purged with 50 ml/min of nitrogen. A hermetic aluminium, closed pan with hole was used, and the sample mass was about 5 mg.

### EXAMPLES

The starting material Elafibranor crude may be obtained according to U.S. Pat. No. 7,943,661. Following filtration, the Elafibranor may be optionally dried to remove residual solvent, e.g. suction dried on the filter, air dried, or vacuum dried (e.g. at room temperature).

### Example 1: Preparation of Elafibranor Form I

Crude Elafibranor (50 mg) was dissolved in EtOH, abs. (1 mL) at about 50° C. Clear solution was left at room temperature for 1 day to crystallize. Obtained solid was filtered. Elafibranor form I has been confirmed by PXRD as presented in FIG. 1.

### Example 2: Preparation of Elafibranor Form II

Crude Elafibranor (50 mg) was dissolved in Ethylbenzene (1 mL) at about 130° C. Clear solution was left at room temperature for 1 day to crystallize. Obtained solid was filtered. Elafibranor form II has been confirmed by PXRD as presented in FIG. 2.

### Example 3: Preparation of Elafibranor Form III

Crude Elafibranor (50 mg) was dissolved in p-Xylene (1 mL) at about 130° C. Clear solution was left at room temperature for 1 day to crystallize. Obtained solid was filtered. Elafibranor form III has been confirmed by PXRD as presented in FIG. 3.

### Example 4: Preparation of Elafibranor Form IV

Crude Elafibranor (150 mg) was dissolved in Acetic acid (3 mL) at about 90° C. Sealed flask with clear solution was left at room temperature to crystallize. After 3 days, flask was left open at room temperature. Solvent evaporated after 1 day. Obtained solid Elafibranor form IV has been confirmed by PXRD as presented in FIG. 4.

### Example 5: Preparation of Elafibranor Form V

Crude Elafibranor (46 mg) was dissolved in Dimethylacetamide (0.22 mL) at room temperature. Sample started to crystallize by water addition (1 mL). Suspension was left at room temperature and filtered after 3 days over the white ribbon. Obtained solid Elafibranor form V has been confirmed by PXRD as presented in FIG. 5.

### Example 6: Preparation of Elafibranor Form VI

Crude Elafibranor (45 mg) was dissolved in N-Methyl-2-pyrrolidone (0.23 mL) at room temperature. Diisopropyl ether (1 mL) was added drop by drop. Sealed flask with clear solution was left at room temperature to crystallize. Solution crystallized after 1 day. Obtained solid was filtered over the white ribbon. Elafibranor form VI has been confirmed by PXRD as presented in FIG. 6.

### Example 7: Preparation of Elafibranor Form VII

Crude Elafibranor (1.0 g) was dissolved in 2-PrOH (50 mL) at 38° C. Solution was filtered over the white ribbon and added dropwise in 150 mL of water at 0° C. Obtained solid was filtered over the white ribbon with vacuum. Elafibranor form VII has been confirmed by PXRD as presented in FIG. 7.

Further aspects and embodiments of the present invention are set out in the following numbered clauses 1A-20A and 1B-20B:

1A. A crystalline Elafibranor designated as Form I, characterized by data selected from one or more of the following:

(a) having a PXRD pattern having peaks at 7.8, 16.3, 17.2, 24.4 and 28.3 degrees 2-theta±0.1 degrees 2-theta; or

(b) an XRPD pattern as depicted in FIG. 1; and

(c) combinations of these data.

2A. Crystalline Elafibranor according to Clause 1A having one, two, three, four or five additional peaks at 11.2, 15.3, 21.4, 25.3 and 27.8 degrees 2-theta±0.1 degrees 2-theta.

3A. Crystalline Elafibranor according to Clause 1A or Clause 2A having a melting point peak at about 154° C. to about 164° C., preferably at about 158° C. to about 160° C., and most preferably at about 159° C. as measured by DSC, and preferably a DSC thermogram having an endothermic peak with onset in the range of about 140° C. to about 160° C., more preferably in the range of about 150° C. to about 158° C., in particular at about 154° C.; and particularly has a DSC thermogram substantially as depicted in FIG. 8.

4A. Crystalline Elafibranor according to any one of Clauses 1A-3A, which is an anhydrous form.

5A. Crystalline Elafibranor according to any one of Clauses 1A-4A, which is substantially free of any other solid state form of Elafibranor.

6A. Crystalline Elafibranor according to any one of Clauses 1A-5A, which is non-hygroscopic, preferably wherein the crystalline form shows less than 0.1% (w/w) of water absorption after exposure to 25° C./80% RH for 24 hours.

7A. Use of crystalline Elafibranor according to any one of Clauses 1A-6A, for preparing other solid state forms of Elafibranor.

8A. Use of crystalline Elafibranor according to any one of Clauses 1A-6A for the preparation of a pharmaceutical composition or formulation, preferably wherein the pharmaceutical composition or formulation is for treating nonalcoholic steatohepatitis.

9A. Crystalline Elafibranor according to any one of Clauses 1A-6A for use as a medicament, preferably for the treatment of nonalcoholic steatohepatitis.

10A. Crystalline Elafibranor according to any one of Clauses 1A-6A for use in the preparation of a pharmaceutical composition or formulation, preferably for the treatment of nonalcoholic steatohepatitis.

11A. A pharmaceutical composition or formulation comprising crystalline Elafibranor according to any one of Clauses 1A-6A.

12A. A pharmaceutical composition or formulation according to Clause 11A comprising at least one pharmaceutically acceptable excipient, preferably wherein the pharmaceutical composition or formulation is for oral administration, and more preferably wherein the pharmaceutical composition or formulation is in a form of tablet or capsule.

13A. A process for preparing a pharmaceutical composition or formulation according to any one of Clauses 11A-12A, comprising combining crystalline Elafibranor according to any one of Clauses 1A-6A, and at least one pharmaceutically acceptable excipient.

14A. A process for preparing other solid state forms of Elafibranor comprising preparing crystalline Elafibranor according to any one of Clauses 1A-6A and converting it to a different solid state forms of Elafibranor.

15A. A method of treating nonalcoholic steatohepatitis, comprising administering a therapeutically effective amount of the crystalline Elafibranor according to any one of Clauses 1A-6A, or a pharmaceutical composition or formulation according to any one of Clauses 11A-12A to a subject suffering from nonalcoholic steatohepatitis, or otherwise in need of the treatment.

16A. A process for preparing a crystalline form of Elafibranor comprising crystallising Elafibranor from a solvent comprising ethanol, preferably wherein the ethanol contains: <10 wt %, preferably <5 wt %, more preferably <2 wt %, or particularly <1% wt % water, and most preferably wherein the solvent is absolute ethanol; optionally isolating and optionally drying the crystalline Elafibranor.

17A. A process according to Clause 16A, comprising dissolving Elafibranor in the solvent, preferably at a temperature of: 20-80° C., preferably 35-70° C., more preferably 40-60° C., or most preferably about 50° C. to form a solution, and crystallizing the Elafibranor from the solution.

18A. A process according to Clause 17A, wherein the crystallizing comprises allowing the solution of Elafibranor in the solvent to cool, preferably to room temperature, and allowing the solution to stand for: 6-48 hours, preferably 12-30 hours and more preferably 18-28 hours.

19A. A process according to any of Clauses 16A-18A further comprising combining the Elafibranor with at least one pharmaceutically acceptable excipient to form a pharmaceutical composition or formulation.

20A. Crystalline Elafibranor obtainable by a process according to any of Clauses 16A-18A, or a pharmaceutical composition or formulation obtainable by a process according to Clause 19A.

1B. A crystalline Elafibranor designated as Form II, characterized by data selected from one or more of the following:

(a) having a PXRD pattern having peaks at 10.9, 23.8, 24.3 and 28.1 degrees 2-theta±0.1 degrees 2-theta; or

(b) an XRPD pattern as depicted in FIG. 2; and

(c) combinations of these data.

2B. Crystalline Elafibranor according to Clause 1B having one, two, three or four additional peaks at 15.6, 21.2, 25.0 and 27.6 degrees 2-theta±0.1 degrees 2-theta.

3B Crystalline Elafibranor according to Clause 1B or Clause 2B having DSC thermogram which exhibits an endothermic event with onset in the range of about 127° C. to about 155° C., preferably in the range of about 130° C. to about 150° C., and more preferably exhibits two consecutive endothermic events with onsets in the range of about 137° C. and about 145° C. as measured by DSC with heating rate of 10° C., and preferably a DSC thermogram substantially as depicted in FIG. 9.

4B. Crystalline Elafibranor according to any one of Clauses 1B-3B, which is an anhydrous form.

5B. Crystalline Elafibranor according to any one of Clauses 1B-4B, which is substantially free of any other solid state form of Elafibranor.

6B. Crystalline Elafibranor according to any one of Clauses 1B-5B, which is non-hygroscopic, preferably wherein the crystalline form shows less than 0.1% (w/w) of water absorption after exposure to 25° C./80% RH for 24 hours.

7B. Use of crystalline Elafibranor according to any one of Clauses 1B-6B, for preparing other solid state forms of Elafibranor.

8B. Use of crystalline Elafibranor according to any one of Clauses 1B-6B for the preparation of a pharmaceutical composition or formulation, preferably wherein the pharmaceutical composition or formulation is for treating nonalcoholic steatohepatitis.

9B. Crystalline Elafibranor according to any one of Clauses 1B-6B for use as a medicament, preferably for the treatment of nonalcoholic steatohepatitis.

10B. Crystalline Elafibranor according to any one of Clauses 1B-6B for use in the preparation of a pharmaceutical composition or formulation, preferably for the treatment of nonalcoholic steatohepatitis.

11B. A pharmaceutical composition or formulation comprising crystalline Elafibranor according to any one of Clauses 1B-6B.

12B. A pharmaceutical composition or formulation according to Clause 11B comprising at least one pharmaceutically acceptable excipient, preferably wherein the pharmaceutical composition or formulation is for oral administration, and more preferably wherein the pharmaceutical composition or formulation is in a form of tablet or capsule.

13B. A process for preparing a pharmaceutical composition or formulation according to any one of Clauses 11B-12B, comprising combining crystalline Elafibranor according to any one of Clauses 1B-6B, and at least one pharmaceutically acceptable excipient.

14B. A process for preparing other solid state forms of Elafibranor comprising preparing crystalline Elafibranor according to any one of Clauses 1B-6B and converting it to a different solid state forms of Elafibranor.

15B. A method of treating nonalcoholic steatohepatitis, comprising administering a therapeutically effective amount of the crystalline Elafibranor according to any one of Clauses 1B-6B, or a pharmaceutical composition or formulation according to any one of Clauses 11B-12B to a subject suffering from nonalcoholic steatohepatitis, or otherwise in need of the treatment.

16B. A process for preparing crystalline Elafibranor comprising crystallising Elafibranor from a solvent comprising ethylbenzene; optionally isolating and optionally drying the crystalline Elafibranor.

17B. A process according to Clause 16B, comprising dissolving Elafibranor in the solvent, preferably wherein the solvent is ethylbenzene, preferably at a temperature of: 20-80° C., preferably 35-70° C., more preferably 40-60° C., or most preferably about 50° C. to form a solution, and crystallizing the Elafibranor from the solution.

18B. A process according to Clause 17B, wherein the crystallizing comprises allowing the solution of Elafibranor in the solvent to cool, preferably to room temperature, and allowing the solution to stand for: 6-48 hours, preferably 12-30 hours and more preferably 18-28 hours.

19B. A process according to any of Clauses 16B-18B further comprising combining the Elafibranor with at least one pharmaceutically acceptable excipient to form a pharmaceutical composition or formulation.

20B. Crystalline Elafibranor obtainable by a process according to any of Clauses 16B-18B, or a pharmaceutical composition or formulation obtainable by a process according to Clause 19B.

1. A crystalline Elafibranor having a PXRD pattern having peaks at 7.8, 12.3, 13.5, 15.9 and 22.7 degrees 2-theta±0.2 degrees 2-theta.

2. Crystalline Elafibranor according to claim 1 having one, two, three or four additional peaks at 15.7, 16.3, 17.1, and 23.4 degrees 2-theta±0.2 degrees 2-theta.

3. Crystalline Elafibranor according to claim 1, which is an anhydrous form.

4. Crystalline Elafibranor according to claim 1, which is substantially free of any other solid state form of Elafibranor.

5. Crystalline Elafibranor according to claim 1, which is non-hygroscopic.

6. Crystalline Elafibranor according to claim 1, wherein the crystalline form shows less than 0.1% (w/w) of water absorption after exposure to 25° C./80% RH for 24 hours.

7.-9. (canceled)

10. A pharmaceutical composition or formulation comprising crystalline Elafibranor according to claim 1.

11. A pharmaceutical composition or formulation according to claim 10 comprising at least one pharmaceutically acceptable excipient, and wherein the pharmaceutical composition or formulation is for oral administration.

12. A process for preparing a pharmaceutical composition or formulation according to claim 10, comprising combining crystalline Elafibranor and at least one pharmaceutically acceptable excipient.

13. A process for preparing a different solid state form of Elafibranor comprising preparing crystalline Elafibranor according to claim 1 and converting it to the different solid state form of Elafibranor.

14. A method of treating nonalcoholic steatohepatitis,
comprising administering a therapeutically effective amount of the crystalline Elafibranor according to claim 1 to a subject suffering from nonalcoholic steatohepatitis, or otherwise in need of the treatment.

15. A method of treating nonalcoholic steatohepatitis, comprising administering the pharmaceutical composition or formulation of claim 10 to a subject suffering from nonalcoholic steatohepatitis, or otherwise in need of the treatment.

16. A pharmaceutical composition or formulation according to claim 10 comprising at least one pharmaceutically acceptable excipient, and wherein the pharmaceutical composition or formulation is in the form of a tablet or capsule.
